(19) United States
(12) Patent Application Publication
(10) Pub. No.: US 2026/0191955 A1
(43) Pub. Date: Jul. 9, 2026

(54) COMPOSITION FOR ENHANCING RADIATION-INDUCED ABSCOPAL EFFECT INCLUDING LAG-3 INHIBITOR AND PD-1 INHIBITOR

(71) Applicant: SEOUL NATIONAL UNIVERSITY R&DB FOUNDATION, Seoul (KR)

(72) Inventors: In Ah Kim, Seongnam-si (KR); Yoomin Kim, Uiwang-si (KR); Seung Hyuck Jeon, Seoul (KR); Seongmin Kim, Seoul (KR); Nawon Park, Seongnam-si (KR)

(73) Assignee: SEOUL NATIONAL UNIVERSITY R&DB FOUNDATION, Seoul (KR)

(21) Appl. No.: 19/440,138

(22) Filed: Jan. 5, 2026

(30) Foreign Application Priority Data
Jan. 7, 2025 (KR) 10-2025-0002020

(51) Int. Cl. A61K 39/395; A61K 39/00; A61N 5/10; A61P 35/00

(52) U.S. Cl. CPC A61K 39/3955; A61N 5/10; A61P 35/00; A61K 2039/507; A61N 2005/1098

(57) ABSTRACT

Disclosed herein is a composition for enhancing an abscopal effect of radiation, including a lymphocyte activation gene-3 (LAG-3) inhibitor and a programmed cell death protein-1 (PD-1) inhibitor. One aspect relates to a method of enhancing an abscopal effect of radiation by co-administering a composition including an LAG-3 inhibitor and a PD-1 inhibitor in conjunction with radiotherapy in the treatment of cancer (e.g., triple-negative breast cancer).

### CROSS-REFERENCE TO RELATED APPLICATION

This application claims priority to and the benefit of Korean Patent Application No. 10-2025-0002020, filed on Jan. 7, 2025, the disclosure of which is incorporated herein by reference in its entirety.

### BACKGROUND

### 1. Field of the Invention

The present specification discloses a composition that enhances an abscopal effect of radiation, including a lymphocyte activation gene-3 (LAG-3) inhibitor and a programmed cell death protein-1 (PD-1) inhibitor. More specifically, the present specification discloses a method of enhancing an abscopal effect of radiation by co-administering a composition including an LAG-3 inhibitor and a PD-1 inhibitor in conjunction with radiation therapy for the treatment of cancer (e.g., triple-negative breast cancer).

### 2. Discussion of Related Art

The abscopal effect is a systemic effect of radiotherapy, which refers to the phenomenon in which treatment effects are shown even in non-irradiated metastatic tumors. Studies have reported that a radiotherapy schedule of 8 GyΔ3 fractions may most effectively induce systemic anti-tumor immune responses and the abscopal effect of radiation when combined with immune checkpoint inhibitors [Vanpouille-Box et al., DNA exonuclease Trex1regulates radiotherapy-induced tumor immunogenicity, Nature Communications (2017); Sandra Demaria et al., Radiation dose and fraction in immunotherapy, Journal for ImmunoTherapy (2021)]. However, the abscopal effect is rarely observed in actual preclinical and clinical studies.

While radiotherapy may induce the activation of anti-tumor T cells, radiotherapy alone may not be able to induce a sufficient systemic anti-tumor immune response. Furthermore, radiotherapy may also activate immunosuppressive cells, including regulatory T cells, and therefore, controlling these immunosuppressive cells is essential to maximizing the effects of radiotherapy-induced immune responses. Cancer cells utilize various mechanisms to evade immune responses, and immunosuppressive cells play a crucial role in the tumor microenvironment surrounding malignant tumors. Therefore, in order to maximize the abscopal effect of radiotherapy, employing strategies to appropriately control immunosuppressive cells is important. Recently, the possibility that the combined use of drugs and therapies targeting immunosuppressive cells in the tumor microenvironment may enhance the abscopal effect has been raised. This has led to increased attention on novel approaches that integrate radiotherapy and immunotherapy in cancer treatment.

Accordingly, the present inventors confirmed that the combined use of a next-generation immunotherapy agent, an LAG-3 inhibitor, and an existing PD-1 inhibitor in radiotherapy may significantly increase the systemic anti-tumor immune response and significantly enhancing the abscopal effect of radiation, which is manifested by tumor growth inhibition and metastasis reduction, thereby completing the present invention.

### SUMMARY OF THE INVENTION

One objective of the present invention is to provide a composition or method for enhancing the abscopal effect of radiation.

To achieve the above objective, the present invention provides, in one aspect, a composition for enhancing an abscopal effect of radiation, including a lymphocyte activation gene-3 (LAG-3) inhibitor and a programmed cell death protein-1 (PD-1) inhibitor.

In another aspect, the present invention provides a method of enhancing an abscopal effect of radiation, including a step of administering the composition and a step of irradiating.

One aspect of the present invention relates to a triple combination therapy in which radiotherapy is added to an immunotherapy combination therapy using an LAG-3 inhibitor and a PD-1 inhibitor. This therapy can effectively suppress the growth of not only irradiated tumors but also non-irradiated tumors, and can effectively suppress and block tumor metastasis. Furthermore, it can maximize the abscopal effect of radiation by activating anti-tumor immune cells within tumor tissues, thereby promoting a systemic anti-tumor immune response. This can be utilized to prevent or treat intractable cancers such as triple-negative breast cancer, and can suggest a new treatment strategy for various cancer types. The present invention is expected to play a crucial role in increasing the effectiveness of cancer treatment and improving patient survival rates by contributing to overcoming immune evasion mechanisms that existing single-agent therapies have limitations in addressing.

### BRIEF DESCRIPTION OF THE DRAWINGS

FIG. 1 illustrates an immune response mechanism utilizing radiotherapy (RT), an immune checkpoint inhibitor (a programmed cell death protein-1 (PD-1) inhibitor), and a lymphocyte activation gene-3 (LAG-3) inhibitor in tumor treatment. Radiotherapy directly destroys tumor cells, thereby inducing an immune response at the tumor site, resulting in a tumor vaccine effect. A PD-1 inhibitor activates CD8+ cytotoxic T cells to attack tumor cells, and an LAG-3 inhibitor inactivates immunosuppressive cells (T_{reg}) to enhance CD8+ T cell activity. In particular, LAG-3 is one of the immunosuppressive substances known to suppress T cell activation and cytokine secretion, exerting inhibitory effects on various types of T cells. Inhibiting this pathway can simultaneously induce the activation of anti-tumor T cells and the suppression of regulatory T cell (Treg) function. Therefore, adding an existing PD-1 inhibitor and an LAG-3 inhibitor to radiotherapy can effectively enhance the abscopal effect by suppressing the activity of immunosuppressive cells even in non-irradiated tumors and increasing the number and activity of anti-tumor immune cells, including CD8+ T cells. This combination therapy contributes to inducing a more potent and systemic anti-tumor immune response by modulating the tumor immune microenvironment.

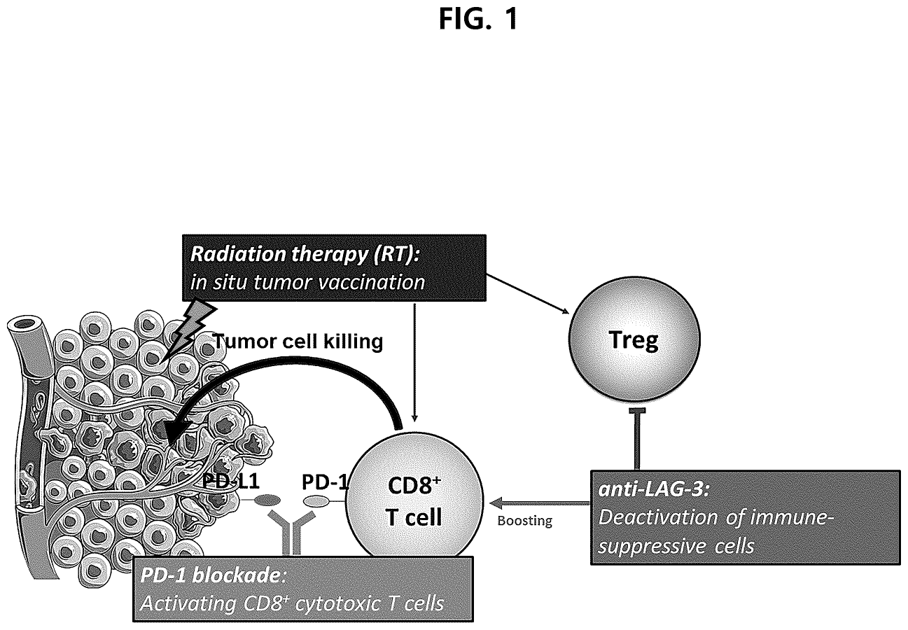

FIG. 2 is a diagram illustrating an animal model experiment designed to evaluate the combined effects of LAG-3 inhibitor and PD-1 inhibitor treatment with radiotherapy (RT).

FIG. 3 is a diagram illustrating the results of analyzing the growth of the irradiated tumor (primary tumor, right hind limb) in the animal model of FIG. 2.

FIG. 4 is a diagram illustrating the results of analyzing the growth of the non-irradiated tumor (secondary tumor, left flank) in the animal model of FIG. 2.

FIG. 5 is a diagram illustrating the results of analyzing the number of tumor nodules metastasized to lung tissue in the animal model of FIG. 2.

FIG. 6 is a diagram comparing the changes in immune cell distribution within the non-irradiated secondary tumor.

FIG. 7 is a diagram illustrating the results of analyzing the distribution of immune cells within the spleen, which represents a systemic immune response.

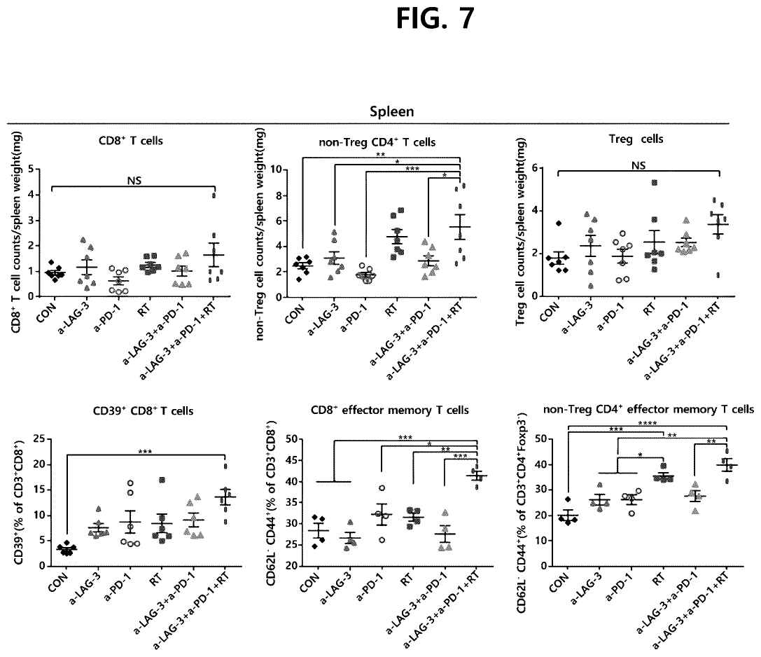

FIG. 8 is a diagram comparing the changes in immune cell distribution within the irradiated primary tumor.

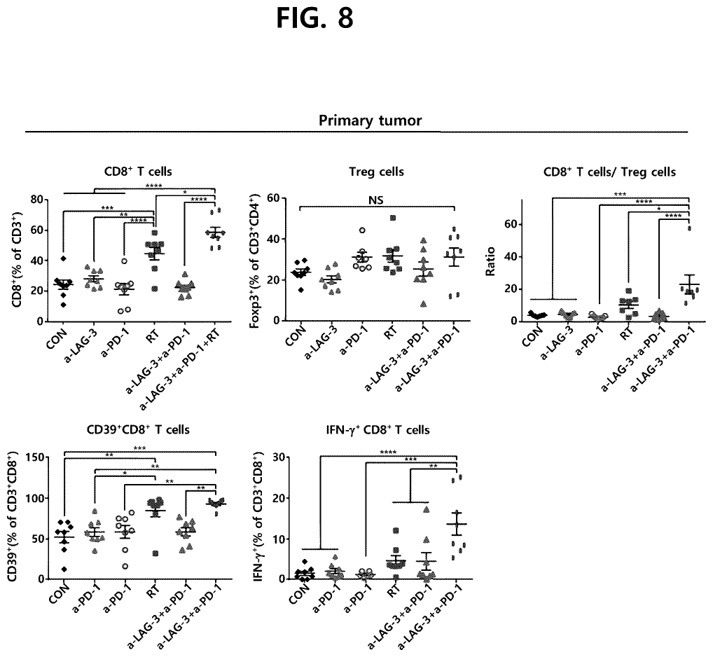

### DETAILED DESCRIPTION OF EXEMPLARY EMBODIMENTS

Hereinafter, the present invention is described in detail.

The term “triple-negative breast cancer” as used herein refers to breast cancer in which the expression of estrogen receptor (ER), progesterone receptor (FR), and HER2 is all negative in immunohistochemical staining. The term may include or be used interchangeably with basal-like breast cancer (BLBC).

In the present invention, the term “abscopal effect” refers to “a biological effect observed at a site remote from a site directly irradiated by radiotherapy.” This includes immunological or cytological responses occurring at sites physically distant from the irradiated site. This is associated with immune system activation and may lead to changes in the tumor microenvironment and systemic immune response.

One aspect of the present invention relates to a composition for enhancing an abscopal effect of radiation, including a lymphocyte activation gene-3 (LAG-3) inhibitor and a programmed cell death protein-1 (PD-1) inhibitor.

In one exemplary embodiment, the LAG-3 inhibitor may be any LAG-3 inhibitor known in the art, without limitation. For example, the LAG-3 inhibitor may include relatlimab, or may be a combination of the LAG-3 inhibitor and another immunomodulatory protein inhibitor (e.g., a PD-1 inhibitor).

In one exemplary embodiment, the PD-1 inhibitor may be any PD-1 inhibitor known in the art, without limitation. For example, the PD-1 inhibitor may include one or more selected from the group consisting of pembrolizumab and nivolumab, or a combination of two thereof.

In one exemplary embodiment, the composition may be administered to a subject in need of inhibiting growth of irradiated tumors and/or non-irradiated tumors.

In one exemplary embodiment, the composition may be administered to a subject in need of reducing a number of tumor nodules metastasized to other tissues (e.g., lung tissue).

In one exemplary embodiment, the composition may be administered to a subject in need of activating immune cells in irradiated tumors, non-irradiated tumors, and/or spleen.

In one exemplary embodiment, the composition may be administered to a subject in need of an increase in CD8+ T cells, a decrease in regulatory T cells (T_{reg}), an increase in effector memory T cells (CD8+ and CD4+), and/or an increase in tumor-specific CD8+ T cells (CD39+) in non-irradiated tumors.

In one exemplary embodiment, the composition may be administered to a subject in need of an increase in non-T_{reg} CD4+ T cells, an increase in effector memory T cells (CD8+ and CD4+), and/or an increase in tumor-specific CD8+ T cells (CD39+) in spleen.

In one exemplary embodiment, the composition may be administered to a subject in need of an increase in CD8+ T cells, an increase in tumor-specific CD8+ T cells (CD39+), and/or an increase of interferon gamma (IFN-γ) expression in irradiated tumors.

In an exemplary embodiment, the LAG-3 inhibitor and the PD-1 inhibitor may be included in a mass and/or volume ratio of 1:9 to 9:1 (LAG-3 inhibitor: PD-1 inhibitor, hereinafter the same), a mass and/or volume ratio of 1:5 to 5:1, or a mass and/or volume ratio of 2:5 to 5:2, for example, a mass and/or volume ratio of 1:1. Furthermore, the LAG-3 inhibitor and the PD-1 inhibitor may be administered sequentially to a subject. The concentration of each inhibitor may be within a range typically tolerated for research and/or treatment in the subject and may be extrapolated within the lethal dose range of the drug.

In an exemplary embodiment, the LAG-3 inhibitor and the PD-1 inhibitor may be included in an amount of, 0.1% to 10% by weight, 0.1% to 5% by weight, or 0.1% to 1% by weight of the total composition, but is not limited thereto.

The term “prevention” as used herein refers to any act of suppressing or delaying a disease or condition by administration of the pharmaceutical composition of the present invention in one embodiment, and the term “treatment” refers to any act of ameliorating or beneficially changing the symptoms of a subject suspected of having a disease or condition by administration of the pharmaceutical composition of the present invention in one embodiment.

In one exemplary embodiment, the composition may be a pharmaceutical composition for preventing or treating cancer, preferably triple-negative breast cancer.

In one exemplary embodiment, the pharmaceutical composition may be administered at an appropriate interval, taking into account the patient's age, stage, weight, prognosis, and/or other factors commonly considered in the art during administration. For example, the composition may be administered 1 to 35 times per week, 1 to 21 times per week, 1 to 14 times per week, 1 to 7 times per week, 1 to 5 times per week, 1 to 3 times per week, 1 to 2 times every other day, or a combination thereof.

In one exemplary embodiment, the pharmaceutical composition may be used in combination with radiotherapy for the treatment of triple-negative breast cancer.

In an exemplary embodiment, the radiotherapy may use any radiotherapy method known in the art, and in one example, a radiotherapy method known to have a high possibility of activating the immune system in terms of radiation dose per session may be used. For example, as a radiotherapy range that may be implemented for actual research or treatment, the radiotherapy may be performed at an intensity of 8 to 12 Gy per session, a total of 1 to 5 times (Fx), and an irradiation interval of 1 to 3 days or 2 to 3 days.

In one exemplary embodiment, the pharmaceutical composition may be used in combination with radiotherapy for the alleviation and/or treatment of triple-negative breast cancer (primary tumor), metastases, and/or recurrent tumors thereof.

The term “metastatic tumor” as used herein refers to a tumor that has spread from the primary site of triple-negative breast cancer to another site distant from the primary site, and the term “recurrent tumor” refers to a tumor of the same type as the primary tumor that has developed in the same position and/or a distant position from the primary tumor.

In an exemplary embodiment, the method of administrating the pharmaceutical composition is not particularly limited, and the pharmaceutical composition may be administered using one or more, two or more, or three or more administration methods selected from the group consisting of oral administration, intravenous injection, intramuscular injection, intrathecal injection, subcutaneous injection, sublingual administration, buccal mucosal administration, rectal administration, vaginal administration, ocular administration, auricular administration, nasal administration, inhalation administration, spray administration, dermal administration, and transdermal administration. In one example, the pharmaceutical composition may be administered by injection.

In an exemplary embodiment, the formulation of the pharmaceutical composition may be prepared by selecting an appropriate method for the treatment of triple-negative breast cancer, and may be, for example, a dry syrup, an orally disintegrating tablet, a buccal tablet, an effervescent tablet, powder, a sublingual tablet, a liquid, a tablet, a capsule, a chewable tablet, a mouthwash, an enema, a nasal spray, an ointment, a cream, a lotion, a nasal drop, an eye drop, an ear drop, a vaginal suppository, a patch, an anal suppository, an inhalant (aerosol), an inhalant, or a combination thereof, but is not limited thereto, and an appropriate formulation may be selected and used by considering the weight, age, stage, and/or prognosis of the administration subject.

In an exemplary embodiment, the pharmaceutical composition may further include an additive in addition to the active ingredients, the LAG-3 inhibitor and the PD-1 inhibitor, for the purposes of increasing pharmaceutical utility, facilitating formulation, promoting stabilization of the formulation, and improving appearance. The additive may be an excipient, a stabilizer, a preservative, a buffer, a coagulant, a suspending agent, an emulsifier, a fragrance, a solubilizing agent, a coloring agent, a concentrate, or a combination thereof, as needed. The additive may be freely selected and used within a range that does not exhibit a direct pharmacological action at the dosage of the pharmaceutical composition of the present disclosure, is safe, and does not change the therapeutic effect of the pharmaceutical composition of the present disclosure or interfere with the test.

In an exemplary embodiment, when the pharmaceutical composition is provided in the form of an injection, the additive may include one or more, two or more, three or more, or all of the additives selected from the group consisting of a solvent, a solubilizing agent, a buffer, an isotonic agent, a stabilizer, an antioxidant, a soothing agent, and a suspending agent. The amount and/or type of the additive may be freely selected and used by a person skilled in the art within the scope of the purpose of the present invention within the range known in the art.

Another aspect of the present invention relates to a method of enhancing a radiation-induced abscopal effect, including a step of administering the composition and a step of irradiating.

Another aspect of the present invention relates to a method of enhancing an abscopal effect of radiation, comprising: a step of administering a lymphocyte activation gene-3 (LAG-3) inhibitor and a programmed cell death protein-1 (PD-1) inhibitor; and a step of irradiating.

In one exemplary embodiment, the irradiation (radiotherapy) may be administered simultaneously with or at a time interval from the administration of the composition including the LAG-3 inhibitor and the PD-1 inhibitor. In one example, the radiotherapy and the administration of the inhibitor may be performed on the same or different days.

In one exemplary embodiment, the method may be used for preventing or treating cancer in a subject.

In one exemplary embodiment, the cancer may be selected from any one of triple-negative breast cancer, a metastatic tumor thereof, and a recurrent tumor thereof.

In one exemplary embodiment, the subject may be in need of inhibiting growth of irradiated tumors and/or non-irradiated tumors.

In one exemplary embodiment, the subject may be in need of reducing a number of tumor nodules metastasized to other tissues (e.g., lung tissue).

In one exemplary embodiment, the subject may be in need of activating immune cells in irradiated tumors, non-irradiated tumors, and/or spleen.

In one exemplary embodiment, wherein the subject may be in need of an increase in CD8+ T cells, a decrease in regulatory T cells (T_{reg}), an increase in effector memory T cells (CD8+ and CD4+), and/or an increase in tumor-specific CD8+ T cells (CD39+) in non-irradiated tumors.

In one exemplary embodiment, the subject may be in need of an increase in non-T_{reg} CD4+ T cells, an increase in effector memory T cells (CD8+ and CD4+), and/or an increase in tumor-specific CD8+ T cells (CD39+) in spleen.

In one exemplary embodiment, the subject may be in need of an increase in CD8+ T cells, an increase in tumor-specific CD8+ T cells (CD39+), and/or an increase of interferon gamma (IFN-γ) expression in irradiated tumors.

In an exemplary embodiment, the LAG-3 inhibitor and the PD-1 inhibitor may be included in a mass and/or volume ratio of 1:9 to 9:1 (LAG-3 inhibitor: PD-1 inhibitor, hereinafter the same), a mass and/or volume ratio of 1:5 to 5:1, or a mass and/or volume ratio of 2:5 to 5:2, for example, a mass and/or volume ratio of 1:1. Furthermore, the LAG-3 inhibitor and the PD-1 inhibitor may be administered sequentially to a subject. The concentration of each inhibitor may be within a range typically tolerated for research and/or treatment in the subject and may be extrapolated within the lethal dose range of the drug.

In an exemplary embodiment, the LAG-3 inhibitor and the PD-1 inhibitor may be included in an amount of, 0.1% to 10% by weight, 0.1% to 5% by weight, or 0.1% to 1% by weight of the total composition, but is not limited thereto.

Hereinafter, the components and effects of the present invention will be described in more detail with examples. However, the examples below are provided for illustrative purposes only to aid understanding of the present invention and are not intended to limit the range and scope of the present invention.

### EXAMPLES

### Example 1

### Design of Animal Model Experiment

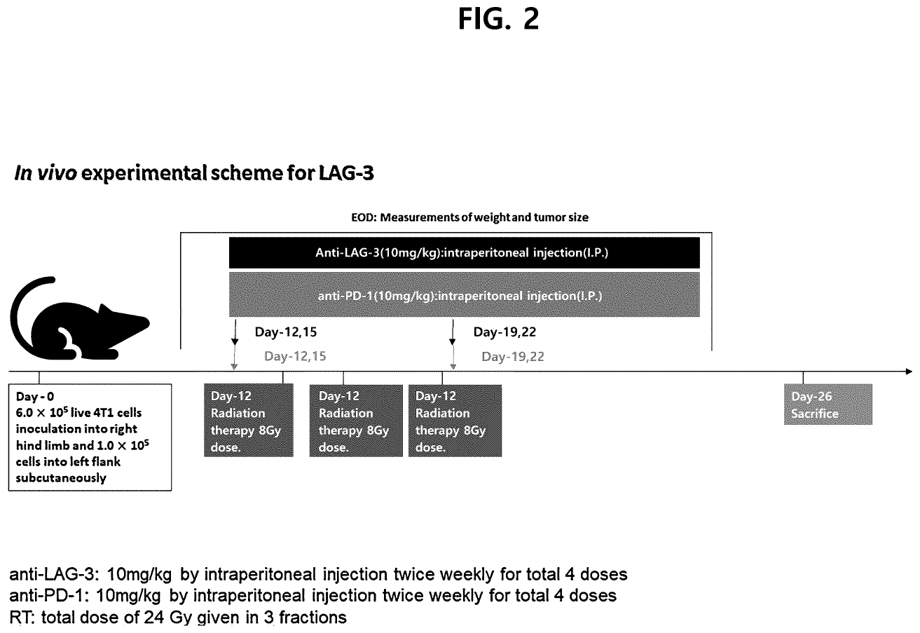

BALB/c mice were implanted with a triple-negative breast cancer cell line (4T1) into the right hind limb (6.0×10⁵ cells, primary tumor) and left flank (1.0×105 cells, secondary tumor). Radiation was administered to the right hind limb tumor alone at 8 Gy×3 fractions for a total of 24 Gy. This radiotherapy schedule activated a systemic antitumor immune response, effectively inducing abscopal effects not only in the irradiated primary tumor but also in the non-irradiated secondary tumors (FIG. 2).

The PD-1 inhibitor was administered intraperitoneally twice a week for a total of four doses, and the LAG-3 inhibitor was administered twice a week for a total of four doses. Tumor sizes were measured every other day, and on day 26 after tumor implantation, mice were sacrificed. Tumor and spleen tissues were extracted and analyzed for changes in the distribution of immune cells within each tissue using flow cytometry. In addition, 4T1 cells have the characteristic of metastasizing to the lungs, and when sacrificing the mice, lung tissue was obtained and the number of metastatic nodules was recorded and analyzed.

### Example 2

### Tumor Growth Inhibition Effect of Combination Therapy With Immunotherapy and Radiotherapy

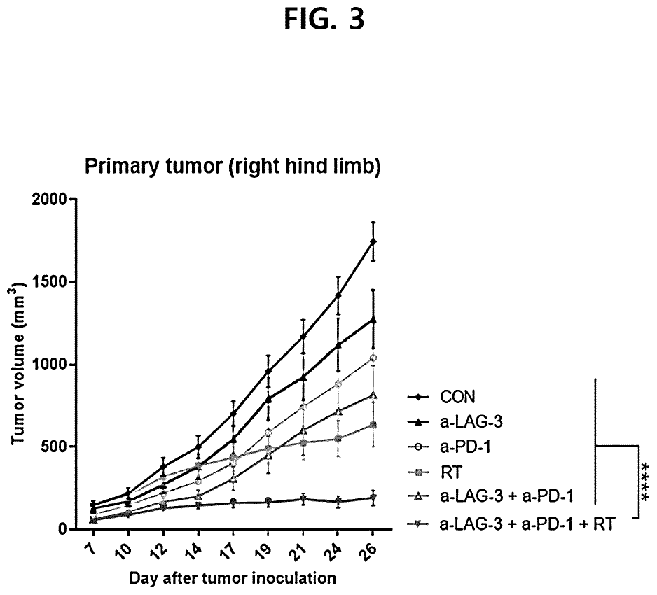

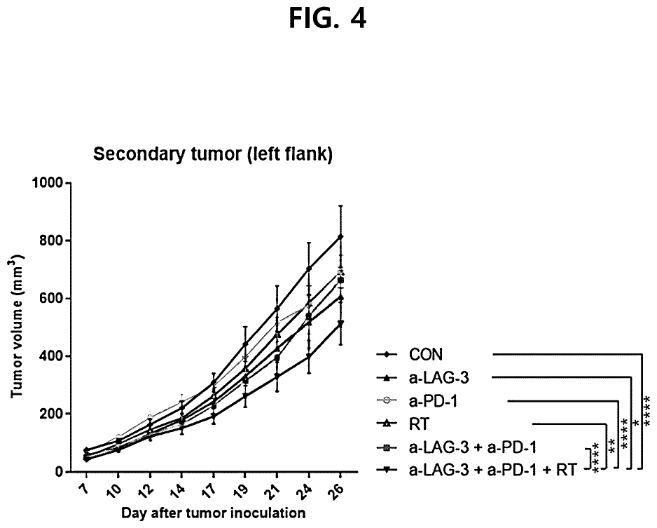

Tumor growth was recorded for the irradiated tumor (primary tumor in the right hind limb) and the non-irradiated tumor (secondary tumor in the left flank). The triple combination therapy of an LAG-3 inhibitor, a PD-1 inhibitor, and radiotherapy demonstrated the most effective inhibition of tumor growth in both groups (FIGS. 3 and 4). Notably, the combination therapy with radiotherapy demonstrated a significantly greater tumor growth delay than the LAG-3 inhibitor and PD-1 inhibitor combination. In the secondary tumors, there was no significant difference in tumor growth between the single treatment groups (LAG-3 inhibitor alone, PD-1 inhibitor alone, or radiotherapy alone) and the LAG-3 inhibitor and PD-1 inhibitor combination group. However, the triple combination therapy group demonstrated the most significant tumor growth reduction, and surprisingly, the most significant abscopal effect was observed in the non-irradiated tumor (secondary tumor in the left flank). In particular, compared to the radiotherapy alone group, the triple combination therapy group, which added a combination of LAG-3 inhibitor and PD-1 inhibitor, showed a significant level of slowing of non-irradiated tumor growth, confirming that the triple combination therapy is very effective in enhancing the abscopal effect.

### Example 3

### Effect of Combination Therapy of Immunotherapy and Radiotherapy in Reducing the Number of Metastatic Tumor Nodules in Lung Tissue

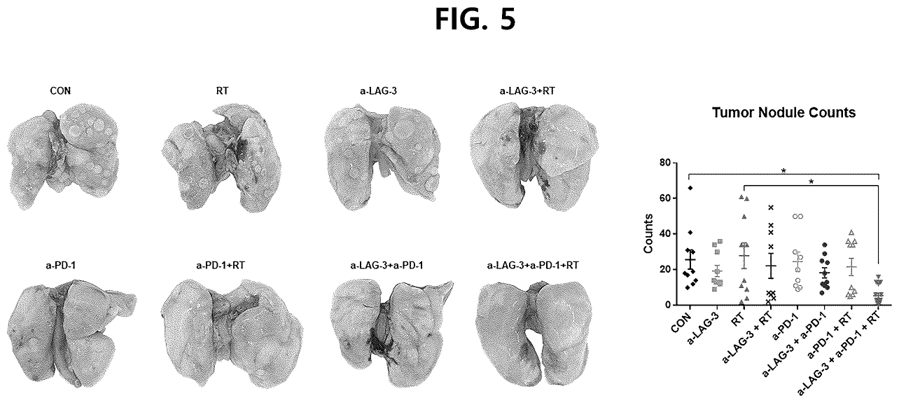

Furthermore, the number of metastatic tumor nodules in lung tissue was examined, and the greatest reduction was observed in the lungs of the triple combination therapy group (FIG. 5). Compared to the radiotherapy alone group, a significant reduction in metastases, that is, an increased abscopal effect, was observed (**p<0.01) . A trend toward a reduction in metastases was observed compared to the LAG-3 and PD-1 combination therapy group

### Example 4

### Changes in Immune Cell Distribution by Combination Therapy With Immune Chemotherapy and Radiotherapy

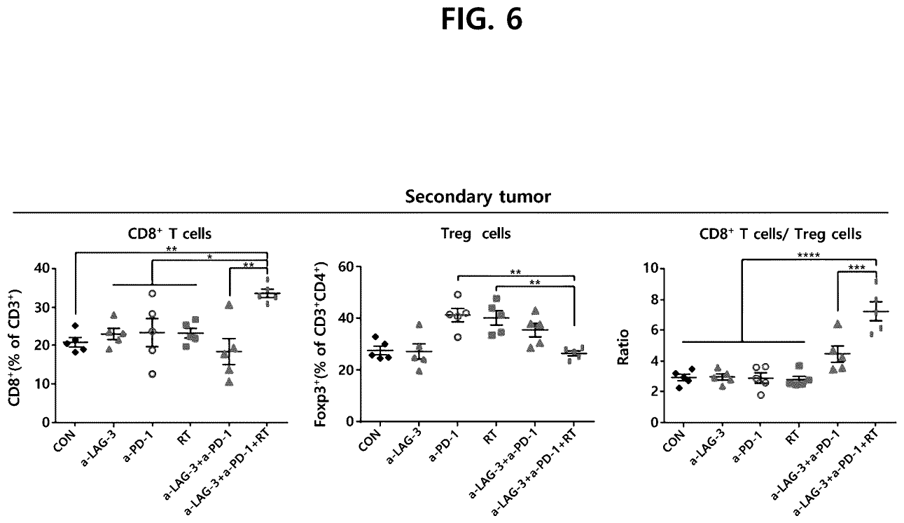

A comparison of immune cell distribution within non-irradiated secondary tumors revealed that the triple combination therapy significantly increased tumor-infiltrating CD8+ T cells, which promote anti-tumor immunity. Regulatory T cells (T_{reg}), which inhibit anti-tumor immunity, increased with radiation alone, but were significantly reduced with the addition of an LAG-3 inhibitor and a PD-1 inhibitor (triple combination therapy). Consequently, the CD8+ T cell/regulatory T cell ratio significantly increased (FIG. 6). These findings suggest that the triple combination therapy significantly enhanced anti-tumor immunity in non-irradiated secondary tumors, and that this likely contributed to the enhanced abscopal effect.

Analysis of the distribution of immune cells in the spleen, which exhibit a systemic immune response, revealed that, among CD8+ T cells and CD4+ T cells, non-T_{reg} CD4+ T cells, which do not express Foxp3, a marker of regulatory T cells, increased in the triple combination therapy group. T_{reg} cells, which inhibit anti-tumor immune responses, showed a slight increase in the triple combination therapy group, but the difference was not significant, and this is thought to be due to radiotherapy and other drug treatments. The proportion of tumor-specific (CD39+) CD8+ T cells increased the most in the triple combination therapy group, and CD8+and CD4+ effector memory T cells, which have antigen-specific memory and are rapidly activated when encountering the same antigen, also increased the most significantly with the triple combination therapy.

In other words, compared to radiation therapy alone or a combination of LAG-3 inhibitor and PD-1 inhibitor, the triple combination therapy can significantly activate the overall anti-tumor immune response in the body, and this surprisingly enhanced the anti-tumor immune function also in non-irradiated secondary tumors.

A comparison of the changes in the distribution of immune cells within the irradiated primary tumor showed that tumor-infiltrating CD8+ T cells, which promote anti-tumor immune function, were significantly increased in the triple combination therapy group compared to the LAG-3 inhibitor and PD-1 inhibitor combination therapy group. Regulatory T cells (T_{reg}), which inhibit anti-tumor immune function, showed a tendency to increase slightly compared to the LAG-3 inhibitor and PD-1 inhibitor combination therapy group, in accordance with previous research results indicating that T_{reg} cells increase when irradiated. However, it was confirmed that the ratio of CD8+ T cells/regulatory T cells was the highest in the triple combination therapy group, which means that although the immunosuppressive properties of T_{reg} cells increased due to the triple combination therapy, the increase in CD8+ T cells was greater than the increase in the T_{reg} cells, and thus the anti-tumor immune response could be most activated. In addition, it was found that CD8+ T cells expressing CD39, a marker indicating tumor specificity, were significantly increased by the triple combination therapy compared to the combination treatment with LAG-3 inhibitor and PD-1 inhibitor, indicating that cytotoxic CD8+ T cells that respond to tumors increased by the triple combination therapy. In addition, it was confirmed that the expression of interferon gamma (IFN-γ), an activation marker of CD8+ T cells that leads to cancer cell death, was significantly increased by the triple combination therapy compared to the combination treatment with LAG-3 inhibitor and PD-1 inhibitor.

In summary of the above-described results, in non-irradiated secondary tumors, compared to the control and single treatment groups, the triple combination therapy group combining LAG-3 inhibitor and PD-1 inhibitor with radiotherapy showed a highly significant tumor growth delay, a significant decrease in the number of tumors metastasizing to the lungs, and activation of anti-tumor immune cells in secondary tumors and spleen tissues. This suggests that combining LAG-3 inhibitor and PD-1 inhibitor with radiotherapy can surprisingly enhance the abscopal effect, leading to inhibition of secondary tumor growth and reduction of metastases. Meanwhile, in the case of irradiated primary tumors, the triple combination therapy group exhibited a very significant decrease in tumor growth compared to the control group, radiotherapy alone group, LAG-3 inhibitor alone group, and PD-1 inhibitor alone group. In particular, a more significant tumor growth inhibition effect was observed in the triple combination therapy group compared to the group using an LAG-3 inhibitor and a PD-1 inhibitor in combination with a statistically highly significant difference (****p<0.0001) , confirming the activation of anti-tumor immune cells in the primary tumor.

This invention relates to a national research and development project supported by the Ministry of Science and ICT (MSIT) and managed by the National Research Foundation of Korea (NRF), under the “Basic Science Research Program (MSIT)” (project title: “Next-generation immune checkpoint inhibitor regulating immunosuppressive cells and combination therapy with radiotherapy”), performed by Seoul National University, where the project period is 2023 Mar. 1 to 2026 Feb. 28 (assignment unique number is 2710017137 and assignment number is 2023R1A2C3003782).

What is claimed is:

1. A method of enhancing an abscopal effect of radiation, comprising:
a step of administering a lymphocyte activation gene-3 (LAG-3) inhibitor and a programmed cell death protein-1 (PD-1) inhibitor; and
a step of irradiating.

2. The method of claim 1, wherein the method is for preventing or treating cancer in a subject.

3. The method of claim 2, wherein the cancer is triple-negative breast cancer, a metastatic tumor thereof, and/or a recurrent tumor thereof.

4. The method of claim 2, wherein the subject is in need of inhibiting growth of irradiated tumors and/or non-irradiated tumors.

5. The method of claim 2, wherein the subject is in need of reducing a number of tumor nodules metastasized to other tissues.

6. The method of claim 2, wherein the subject is in need of activating immune cells in irradiated tumors, non-irradiated tumors, and/or spleen.

7. The method of claim 2, wherein the subject is in need of an increase in CD8+ T cells, a decrease in regulatory T cells (T_{reg}), an increase in effector memory T cells (CD8+ and CD4+), and/or an increase in tumor-specific CD8+ T cells (CD39+) in non-irradiated tumors.

8. The method of claim 2, wherein the subject is in need of an increase in non-T_{reg} CD4+ T cells, an increase in effector memory T cells (CD8+ and CD4+), and/or an increase in tumor-specific CD8+ T cells (CD39+) in spleen.

9. The method of claim 2, wherein the subject is in need of an increase in CD8+ T cells, an increase in tumor-specific CD8+ T cells (CD39+), and/or an increase of interferon gamma (IFN-γ) expression in irradiated tumors.
